(19) United States
(12) Patent Application Publication
(10) Pub. No.: US 2007/0256333 A1
(43) Pub. Date: Nov. 8, 2007

(54) Therapeutic appliance for stress relief

(76) Inventor: Owen Miles Wolf JR., St. Cloud, FL (US)

Correspondence Address: Owen M. Wolf JR., 5400 Alligator LK. Rd., St. Cloud, FL, 34772, US

(21) Appl. No.: 11/389,933

(22) Filed: May 3, 2006

(51) Int. Cl. A43B 13/38; A61F 5/14

(52) U.S. Cl. 36/141; 36/44

(57) ABSTRACT

Massage foot gear enabling wearer to have relaxation and theraputic massage while performing normal walking motion. Continuation of research (medical and technical) to incorporate automatic, non invasive stimulation of atrophied limbs and trunk areas through impulse nodes implanted in flexable wraps. Target meridian points (as in reflexology) in the foot area, to enhance the feeling of well being as well as possible therapy for diseases related to this area. Wraps will also target atrophied muscular areas to increase muscle dexterity and circulation, possibly theraputic for MS patients as well as sport therapies and accident related injuries.

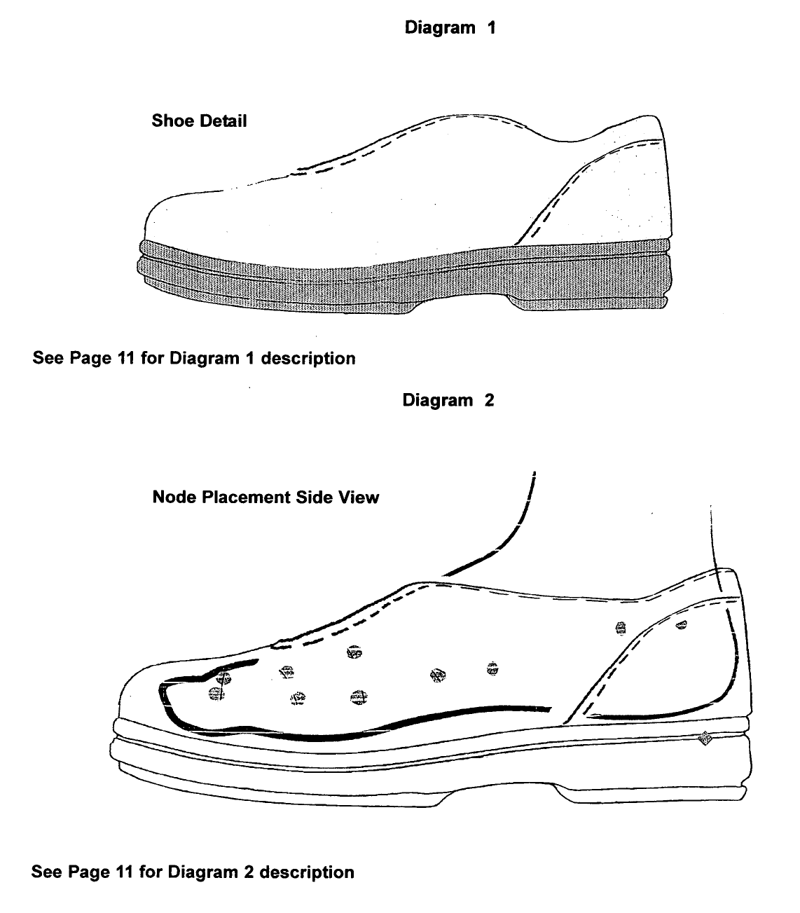

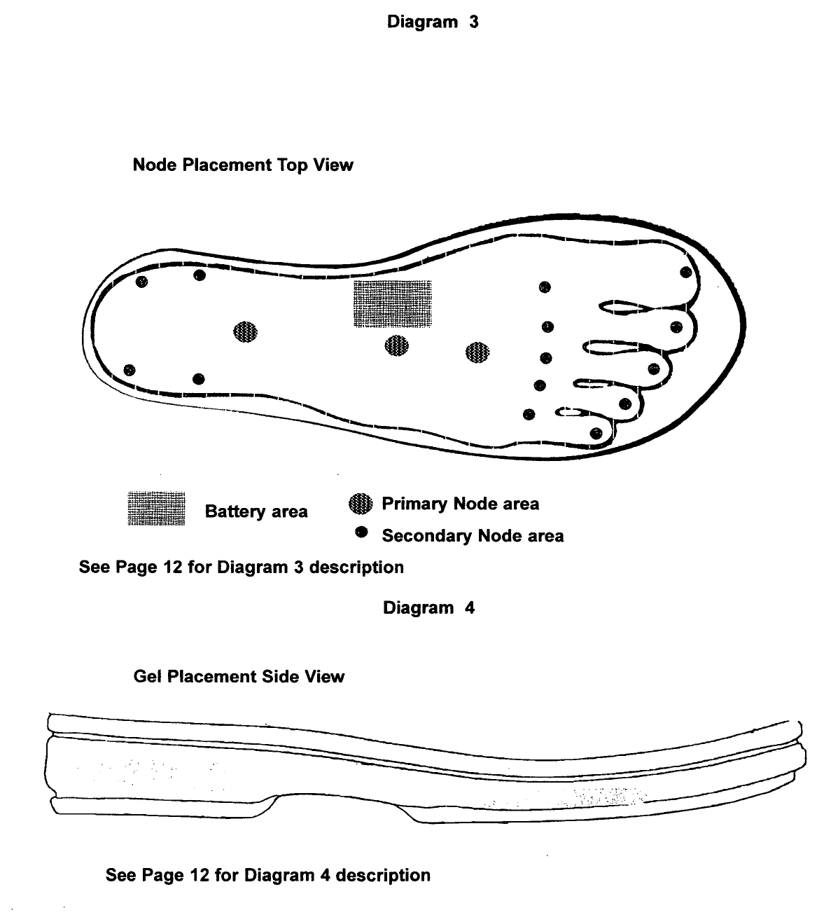

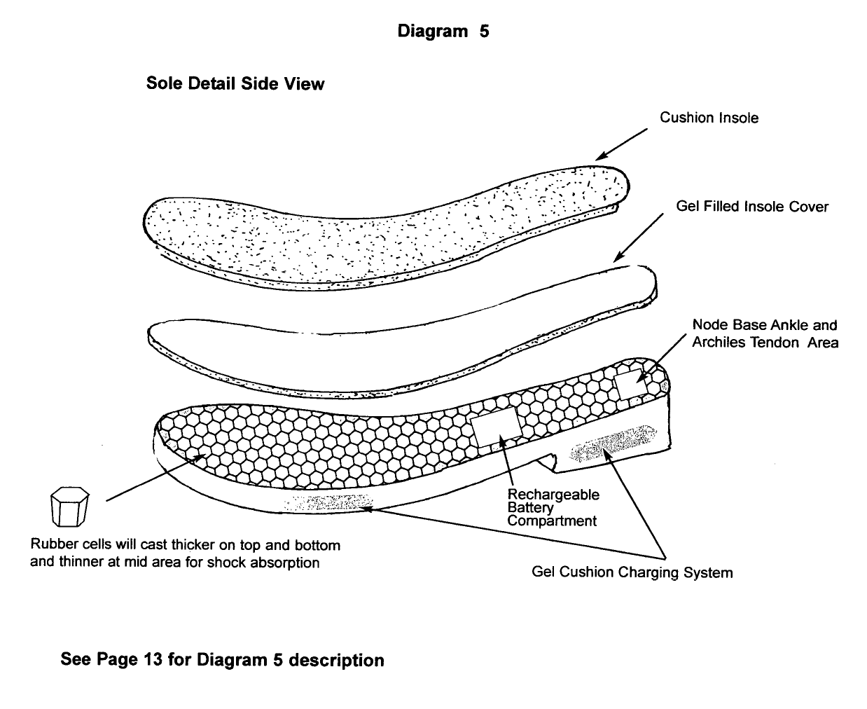

### CONCEPT SCOPE

Footwear containing vibratory nodes that produce impulse massage and stimulation to nerve and pressure points along the oriental 7 meridian lines referenced by reflexology and acupressure charts relative to the health and well being of persons of all health and age groups (Novick A, Birke J A, Brasseaux D M, Broussard J B, Hoard A. Changes in foot posture with varying amounts of weight bearing. Ongoing study). Utilizing a primary node for general relaxation and a secondary node level for therapies. This concept would aid in relaxation, stimulate nerve response and circulation. The concept would then branch out into other areas of concern as in muscle atrophy, natural healing as well as circulatory problems. Research will progress to include Orthopedic, Chiropractic, Internal Medicine and target various diseases that would benefit from increased blood circulation and impulse therapy in a non-invasive unit (Diagram1). (Physical therapy of the foot and ankle. Churchill Livingstone, New York 1988: 133-168).

Concept Highlight:

Therapeutic massage and vibratory relief
Increased blood circulation from stimulation of pressure points
Muscular stimulation to areas of atrophy
Target diseases that can be positively affected by pressure point therapy
Arthritic pain relief
Increased circulatory awareness to relieve atrophy and/or muscle inflammation
Foot and Head areas to be targeted on first release with the development of therapeutic leg/arm wraps, shoes, wristbands and neck wraps using the primary and secondary vibratory node concept.
Incorporate planning/discussions and research into orthopedic problems for resolve through massage and acupressure techniques. (Wu K K. Foot orthosis: principles and clinical applications. Williams and Wilkins, Baltimore 1990.
   Project Details

Electronic (battery source) impulse vibratory nodes are encased in stationary foam cushioned insole and side panels at key therapeutic meridian points powered by an electrical current created by a rechargeable battery installed in a closed compartment in the instep of waterproof rubber honeycomb bottom sole (Diagram5) (Neoprene crepe (Southern Leather Co., New Orleans, La. 70113)). Magnetic field reservoir housed also in base of outer sole would supply the recharge for battery. Copper filament with fluid ground magnetic particles passing through baffled rubber passageway channels contained in base of sole of foot wear provides minute electrical energy for recharge of battery cell.

Primary nodes are placed in 3 key positions (Re: Ontario College of Reflexology, Diagram3) beginning at heel area continuing up through Achilles Tendon to ball of foot area and top of foot utilizing secondary nodes. Lower nodes are encased in honeycomb rubber cells of bottom sole with gel/foamfilled liner topper (Diagram5) (insole made from a single layer of soft, low-durometer material, such as PPT (The Langer Group, Deer Park, N.Y. 11729) or Spenco (Spenco Medical Corp., Waco, Tex. 76710) with the upper and side nodes encased in channels installed into foam filled upper support covered with a layer of soft insole material.

Heel node area continues from 3rd primary node to 4 secondary nodes in circular pattern at heel base to lower ankle and mid heel areas as described (Diagram 2), Ontario College of Reflexology. Impulse flow will start at heel and form continuous loop effect up through the sole of the foot to the top of foot and sequencing again beginning at the heel base. Wave ratio: 1 cycle every 6-8 seconds. Gel base pad on outside of heel area (outer sole, Diagram4) forces ground magnetic particles in a thin oil base solution through connecting elliptical tubes to copper connector wires creating electrical current to maintain constant charge on battery system. Battery is recharged during normal walking movement (Diagram3/5). Fluid is propelled through tubes by pump action created by the heel to ball stepping action.

Beneficiaries:

White Collar Workers

Blue Collar Workers

Athletes

Elderly

Arthritis Patients

Bedridden/injury patients

Orthopedic Therapies

Chiropractic Therapies

General Public all age groups

Social/Economic:

Socially:
Enhanced sense of well being from pressure point massage through stimulation of the 7 oriental meridian points. This system reacts in concept of the current TENS Unit utilizing pressure point therapy for reduction of pain and or stress. (The insensitive foot (including leprosy). In: Jahass M H (ed). Disorders of the foot, vol 2., WB Saunders Co., Philadelphia, 1982; 1266-128).

Economically:
Job opportunities will be created
Community based workforce
On the job training:
   Fabrication
   Production
   Research and development
   Sales
   Marketing
Corporation location to be determined, location preferably in low income or economically depressed area.
   Production Process Analysis:

Patent Update and Renewal with Utilities Draft

Therapy Research

Cost Analysis

Legal Aspects

Consumer Marketing Research

Permitting

Orthopedic Research and Sanctioning
Subsidies targeting Orthopedic, Chiropractic, Natural healing and Podiatry and medical patients

Production/Marketing Analysis

Prototype Production

Target Marketing

Plant Location

Key Points Production Arena

Management Teams

Legal Counsel for Plant Production, Labor

General production/sales teams

National Advertising

Market Placement

### OBJECTIVES

Provide areas of therapeutic massage to specific physical acupressure points for stress relief, increased circulation and muscular deterioration therapies.
Promote awareness in private sector for self induced relief of stress, pain, muscular tensions and natural remedies for thus.
Usage of non-invasive vibratory impulse nodes placed in accordance with proven reflexology and acupressure locations directly affecting specific target areas associated with muscular strains, atrophy and pain.
Controlled sequencing of vibratory nodes to target meridian point system for stimulation to specific internal organs to aid in disease symptom reduction.
Continual research and development of main and subsidy product lines to effectively generate positive therapeutic results in the Orthopedic, Chiropractic and Podiatry medical arenas for muscular atrophy and circulatory benefits for immobilized/injury patients for limb, head/neck and trunk body areas.
   Problem Solving:
Patent revisions on existing patent to incorporate outlined advancement from initial patent granted July 1994.
Physician Consultants for exact node placement and therapy issues to resolve.
Outsource professionals for reflexology node placement for stress relief only version of product line available to general public.
Team assessment of strategies for production.
Management structure for team decision making relating to immediate production and employee issues.
Assessment of strategies used in production placement of modes in relation to relief sites.
Engineering team handling all continuous research and development incorporated in the advancement and production of final product.
Continual medical consultations for therapy issues and advancement of node placements and or wave path configurations.
In house lab and testing for advancement of therapeutic positive results.
Outsource legal and professional medical consultations as required.

1. I, Owen M. Wolf, Jr. as sole inventor of Sole Synergy Foot Gear claim that my invention will relieve stress and tensions in the foot and achilies tendon area creating a feeling of relaxation through the use of vibrating node modules that will sequence massage to the meridian and reflexology points of the foot area.
The technology will then expand to include trunk and limb areas of the anatomy with inclusion of non-invasive medical therapies to induce increased circulation to immobilized or atrophied muscular areas.
